(19) United States
(12) Patent Application Publication
(10) Pub. No.: US 2017/0086560 A1
(43) Pub. Date: Mar. 30, 2017

(54) COSMETIC CASE

(71) Applicant: ZEN DESIGN SOLUTIONS LIMITED, Kowloon (HK)

(72) Inventors: Leo Clifford PIRES, Basking Ridge, NJ (US); Rahul BOSE, New Delhi (IN); Manisha KOHLI, New Delhi (IN)

(21) Appl. No.: 15/269,572

(22) Filed: Sep. 19, 2016

(30) Foreign Application Priority Data
Sep. 24, 2015 (IN) 3031/DEL/2015

(51) Int. Cl. A45D 33/32; A45D 33/00; A45D 40/22; F21V 33/00; A61L 2/10; H05K 7/14; H02J 7/00; A45D 40/18; A45D 33/24

(52) U.S. Cl. CPC A45D 33/32; A45D 40/18; A45D 33/006; A45D 40/222; A45D 33/24; A61L 2/10; H05K 7/1427; H02J 7/0047; F21V 33/004; A61L 2202/11; A61L 2202/14; F21Y 2115/10

(57) ABSTRACT

The present disclosure relates to a cosmetic case, including: a cover, a base, an applicator, a sanitizer which emits ultraviolet rays for sanitizing the applicator, a wave deflector, a power supply for supplying power to the sanitizer, and at least one indicator which is provided to indicate operation conditions of the sanitizer. The sanitizer emits ultra violet rays for a predetermined amount of time for sanitizing the applicator. Further, the cosmetic case includes a micro controller which controls operation conditions of the sanitizer.

### CROSS-REFERENCE TO RELATED APPLICATION

This patent application claims the benefit of priority to Indian Provisional Application Ser. No. 3030/DEL/2015, filed Sep. 24, 2015. The disclosure of the aforementioned patent application is incorporated by reference herein in its entirety for any purpose whatsoever.

### BACKGROUND

Field of the Invention

The present disclosure relates to a cosmetic case with a sanitizer and an applicator, and more particularly, a cosmetic case including an applicator and a sanitizer for sanitizing the applicator.

Description of the Related Art

In general, cosmetics may be divided into fundamental cosmetics such as skin toners, lotions and the like, make-up cosmetics such as powders, compacts and the like, and functional cosmetics for prevention of wrinkles and aging, and their contents are in the form of viscous liquid, solid, powder or gel.

Such cosmetics are stored in a case made of material such as plastics, vinyl, synthetic resin, metal, ceramics or the like.

A cosmetic case storing make-up cosmetics includes a separate applicator for use of powders or foundations in the form of powder or liquid. As used herein, the term “applicator” refers to a make-up tool used to apply cosmetics to skin of a user.

However, an applicator equipped in conventional cosmetic cases contains a quantity of water and oil in the course of contact with a skin, which may provide surroundings favorable for introduction and proliferation of external contaminants such as bacteria or any other micro-organism. Such an applicator contaminated by bacteria or microbes may have an adverse effect on the skin, such as causing a skin trouble or accelerating skin aging.

Accordingly, it is an object of the present disclosure to provide a cosmetic case with an applicator and a sanitizer, which is capable of sanitizing the applicator.

### SUMMARY

To achieve the above object, according to an aspect of the disclosure, there is provided a cosmetic case, including: a cover, a base, an applicator, a sanitizer which emits ultraviolet rays for sanitizing the applicator, a wave deflector, a power supply for supplying power to the sanitizer, and at least one indicator which is provided to indicate operation conditions of the sanitizer. The sanitizer emits ultra violet rays for a predetermined amount of time for sanitizing the applicator. Further, the cosmetic case includes a micro controller which controls operation conditions of the sanitizer.

According to a preferred embodiment of the disclosure, the sanitizer emits ultraviolet rays. According to another embodiment, the sanitizer emits UVC rays with wavelengths between 200-280 nanometers (nm). According to another embodiment, the sanitizer is UVC LED or any other convention source of UVC. The sanitizer disinfects the applicator.

According to another embodiment of the present disclosure, the deflector surrounds the sanitizer to maximize (or amplify) the effects of the UV rays generated from the sanitizer. The deflector can be of any shape but is preferably of frustoconical shape. According to an embodiment of the present disclosure, the wave deflector has a reflective internal surface. Internal surface of the wave deflector can be made reflective by such methods as chrome plating, painting with a light-reflecting paint or by other suitable methods. Alternatively, reflective material such as glass, mirror or highly polished metals can be used for reflective internal surface.

The cover and the base of the cosmetic case are connected by an attachment means in a manner permitting their relative movement with respect to one another to open and close the cosmetic case. The attachment means may comprise a hinge, a pin, a snap, or a hook.

According to another embodiment of the present disclosure, a locking means is provided in the cover and the base to prevent opening of the cosmetic case when not in use. The locking means may comprise of a fastener piece on the cover that matches a fastener unit on the base for securing purpose. Further, a snap, a magnet, a button or any suitable locking means could be provided to secure the cosmetic case.

According to another embodiment of the present disclosure, there is provided a cosmetic case comprising a cover and a base. The base comprises a lower base and an upper base which together define an internal storage space for housing a sanitizer, a wave deflector, at least one indicator to indicate operation conditions of the sanitizer.

According to another embodiment of the present disclosure, the sanitizer and the wave deflector are housed in the internal storage space in the lower base.

According to yet another embodiment of the present disclosure, an applicator is stored in a cavity formed in the upper base. The upper base comprises a side wall and a bottom wall. According to an embodiment, the bottom wall is formed separately from the side wall and then attached to a bottom end of the side wall. In alternate embodiments, the side wall and the bottom wall are made integrally as one unit. Further, at least a portion of the upper base allows the ultra-violet rays emitted by the sanitizer to pass through the internal storage space to the applicator. According to an embodiment, the bottom wall has at least one aperture to allow the ultra-violet rays emitted by the sanitizer to pass through the internal storage space to the applicator. According to an alternate embodiment, at least a portion of the upper base is made of a material which allows ultra-violet rays emitted by the sanitizer to pass through the internal storage space to the applicator. According to an alternate embodiment, at least a portion of the upper base is transparent or translucent to allow the ultra-violet rays emitted by the sanitizer to pass through the internal storage space to the applicator. According to an alternate embodiment, at least a portion of the upper base is opaque with at least one aperture or micro perforation to allow the ultra-violet rays emitted by the sanitizer to pass through the internal storage space to the applicator. According to another alternate embodiment, the bottom wall of the upper base is transparent or translucent to allow the ultra-violet rays emitted by the sanitizer to pass through the internal storage space to the applicator. Further, the side wall of the upper base may be transparent or translucent.

According to yet another embodiment of the present disclosure, the applicator includes at least one applying portion. Preferably, the at least one applying portion faces towards the internal storage space when the applicator is stored in the cavity of the upper base. According to yet another embodiment of the present disclosure, the applicator can be stored in the upper base only when the at least one applying portion faces towards the internal storage space.

The upper base is connected to the lower base by suitable attachment means. According to an embodiment, the upper base has at least one fitment projection extending from the side wall towards the lower base. The at least one fitment projection fits into a corresponding fitment groove in the lower base. According to an alternate embodiment, the upper base may be connected to the lower base by other attachment means including a threaded attachment, snap fit and the like.

The internal storage space further comprises a source of power supply. The power supply includes: a printed circuit board which is stored in the internal storage space and the sanitizer is mounted on the printed circuit board.

According to an embodiment, the printed circuit board is assembled in the internal storage space and on the lower base by suitable attachment means. According to an embodiment, the printed circuit board has at least one cut out which fits into a corresponding at least one projection present in the lower base. According to an alternate embodiment, the printed circuit board may be connected to the lower base by other attachment means including a threaded attachment, snap fit and the like. According to a further embodiment of the present disclosure, a printed circuit board cover is combined to the internal storage space to fix the printed circuit board to the internal storage space. The printed circuit board is further combined with one or more indicators provided to indicate operation conditions of the sanitizer. The one or more indicators are formed with a light emitting diode which are turned on during sanitization of the applicator by the sanitizer and are turned off after completion of the sanitization of the applicator. According to an embodiment, the light emitting diode includes one or more RGB LED which may be a pulsating or a flashing LED.

The internal storage space formed by the upper and the lower base further includes a battery to supply power to the sanitizer. The battery is mounted under the printed circuit board and may be one time use or rechargeable, replaceable or one time use. The base may further include a USB port for charging the battery.

The wave deflector is assembled on the printed circuit board in the internal storage space by suitable attachment means. According to an embodiment, the wave deflector has at least one projection which is inserted into a corresponding at least one guide groove on the printed circuit board. Further, the wave deflector has at least one fitment projection which fits into a corresponding at least one fitment groove on the printed circuit board. According to an alternate embodiment, the wave deflector may be connected to the printed circuit board by other attachment means including a threaded attachment, snap fit and the like.

According to yet another embodiment of the present disclosure, the one or more indicators on the printed circuit board also indicate charging of the at least one battery. The one or more indicators emit different colored lights during sanitization of the applicator, during charging of the battery and when the charging of the battery is complete. Alternatively, the internal storage space may comprise a separate charging indicator which indicates charging of the battery. The charging indicator is also formed with a light emitting diode which is turned on to emit a colored light during charging of the battery and emit a different colored light when the charging of the battery is complete. The light emitting diode includes one or more RGB LED which may be a pulsating or a flashing LED.

The printed circuit board is further provided with a power button which is combined to the attachment means of the cover and the base. According to an embodiment, the printed circuit board is further provided with a power button which is combined to a hinge attaching the cover and the base and the power button is pressed during opening and closing of the cover. The power button is provided to actuate the sanitizer emitting the ultraviolet rays. The sanitizer is actuated for a predetermined amount of time controlled by the micro-controller when the power button is pressed during closing of the cover. The sanitizer is off during opening of the cover.

According to an alternate embodiment, the power button may be replaced by a sensor which functions during opening and closing of the cover. According to an embodiment, the button/sensor is a concealed button/sensor so that it operates only on closing and opening of the cosmetic case and thus the button/sensor cannot be fiddled by the user. The power button can be a magnetic or mechanical button.

According to another embodiment of the present disclosure, after using the applicator, as the cosmetic case is closed, the button/switch/sensor turns on the sanitizer for a predetermined amount of time controlled by the micro-controller and sanitization process is indicated by the operation indicator. The light emitted by the indicator during sanitization is diffused towards the upper base. Accordingly, a user can confirm operation conditions of the sanitization as light emitted during sanitization is visible to user's naked eyes through the transparent or translucent portion of the upper base. Thus, a sanitized applicator will be available to the user during next use of the cosmetic case.

In other embodiments, the operation indicator may be provided in a manner to produce a sound during operation of the sanitizer.

According to another embodiment of the present disclosure, the cosmetic case further comprises a lift tray for storing a cosmetic in a replaceable pan.

The above and other objects, features and advantages of the present disclosure will become clear from the following description of the preferred embodiments when the same is read in conjunction with the accompanying drawings.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 illustrates isometric view of a cosmetic case in a closed state according to one embodiment of the disclosure;

FIG. 2 and FIG. 3 illustrate a front view and a side view respectively of the cosmetic case of FIG. 1;

FIG. 4 illustrates isometric view of the cosmetic case of FIG. 1 in an open state;

FIG. 5 illustrates top view of the cosmetic case of FIG. 1;

FIG. 6 illustrates cross-sectional view along axis B-B of the top view of the cosmetic case of FIG. 5; and

FIG. 7 illustrates exploded view of the cosmetic case of FIG. 3.

To facilitate understanding, identical reference numerals have been used, where possible, to designate identical elements that are common to the figures. It is to be noted, however, that the appended drawings illustrate only typical embodiments of this disclosure and are therefore not to be considered limiting of its scope, for the disclosure may admit to other equally effective embodiments.

### DETAILED DESCRIPTION

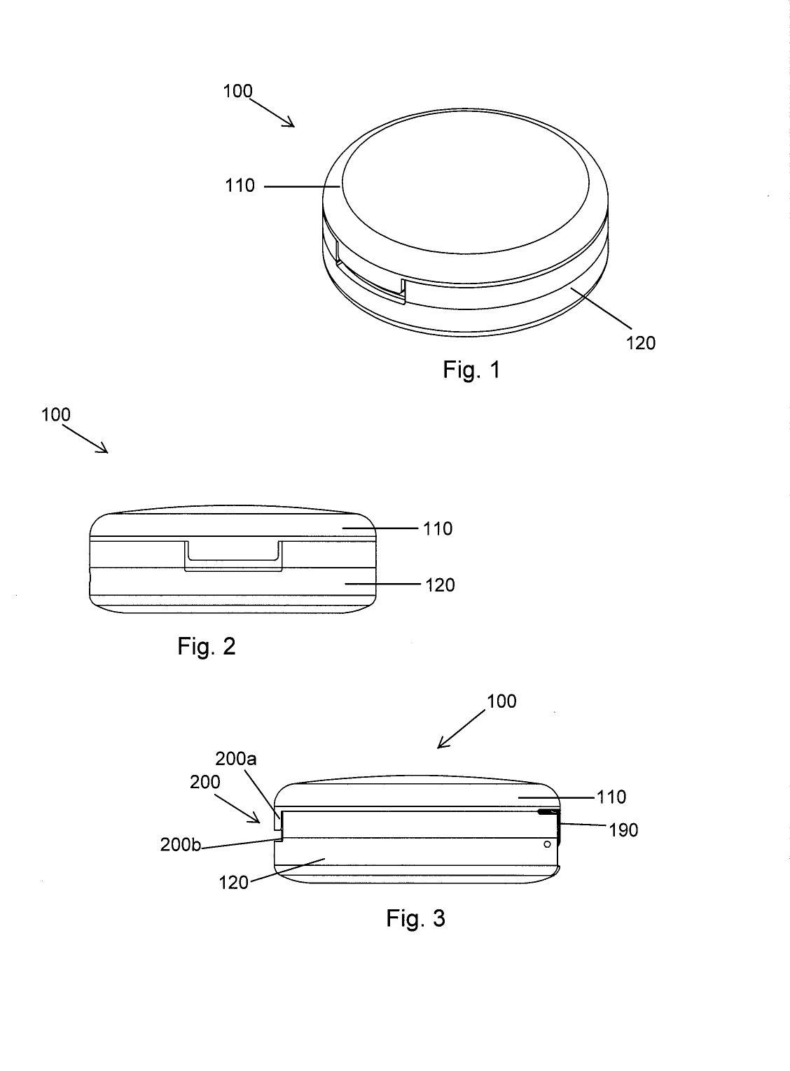

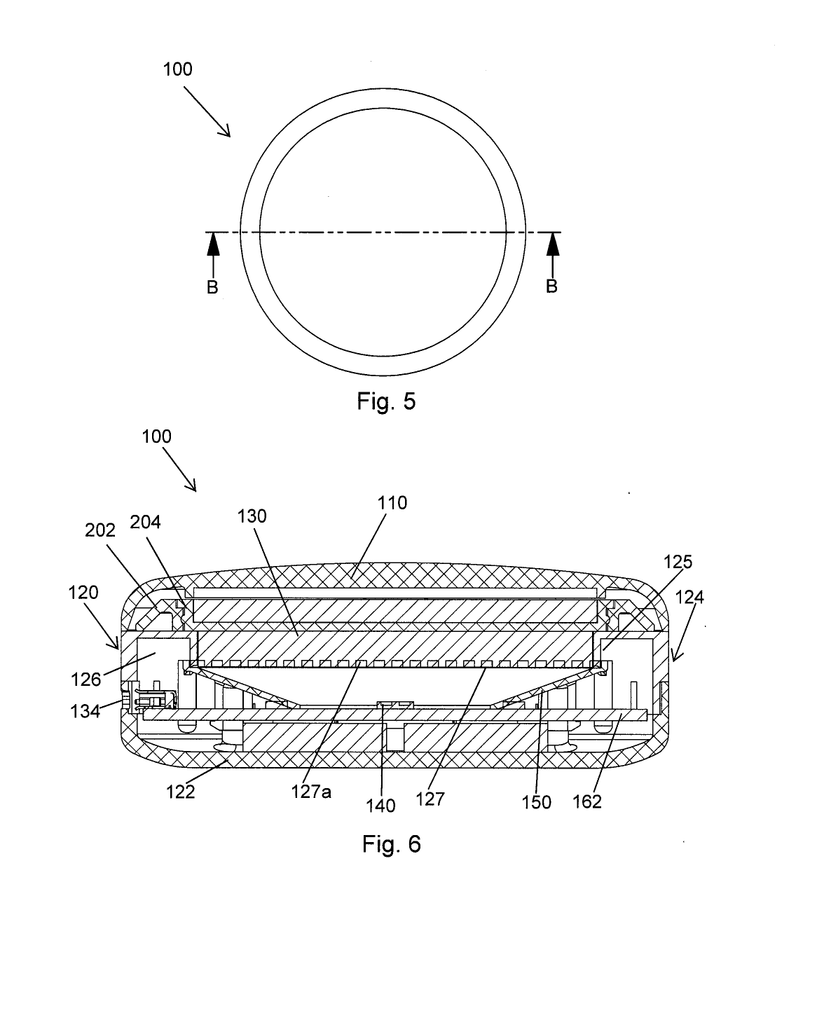

FIGS. 1 through 7 show a cosmetic case 100 according to one embodiment of the present disclosure. The cosmetic case 100 includes a cover 110, a base 120, an applicator 130, a sanitizer 140 which emits ultraviolet rays for sanitizing the applicator 130, a wave deflector 150, a power supply 160 for supplying power to the sanitizer 140, and at least one indicator 170a, 170b which is provided to indicate operation conditions of the sanitizer 140. The sanitizer 140 emits ultra violet rays for a predetermined amount of time for sanitizing the applicator 130. Further, the cosmetic case 100 includes a micro controller 180 which controls operation conditions of the sanitizer 140.

According to a preferred embodiment of the disclosure, the sanitizer 140 includes a UV LED or any other conventional source which emits UVC rays with wavelengths between 200-280 nanometers (nm).

According to another embodiment, the sanitizer 140 disinfects the applicator.

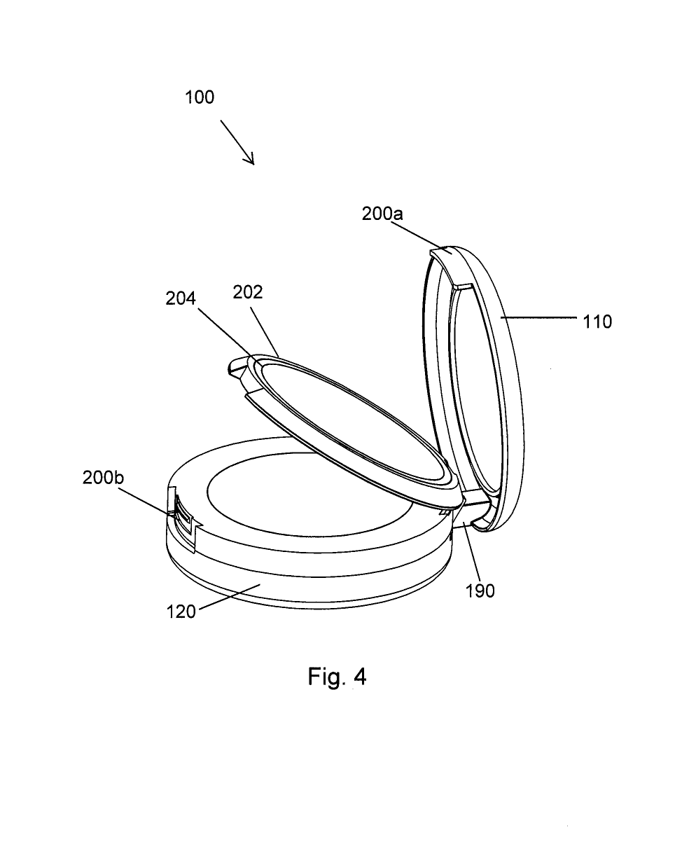

According to present embodiment and as shown in FIG. 4, the cosmetic case 100 further comprises a lift tray 202 for storing a cosmetic in a replaceable pan 204.

Further, as shown in FIG. 6, the wave deflector 150 surrounds the sanitizer 140 which is a UVC LED, to maximize (or amplify) the effects of the UVC rays generated from the sanitizer 140. In the present embodiment, the wave deflector 150 is frustoconical in shape but in other alternate embodiments the wave deflector 150 can be of any shape like circular, square and the like. According to an embodiment of the present disclosure, the wave deflector has a reflective internal surface. Internal surface of the wave deflector 150 can be made reflective by such methods as chrome plating, painting with a light-reflecting paint or by other suitable methods. Alternatively, reflective material such as glass, mirror or highly polished metals can be used for reflective internal surface.

As shown specifically in FIG. 3 and FIG. 4, the cover 110 and the base 120 of the cosmetic case 100 are connected by an attachment means 190 which is a hinge in the present embodiment, in a manner permitting their relative movement with respect to one another to open and close the cosmetic case 100. In other alternate embodiments, the attachment means 190 may comprise a pin, a snap, or a hook.

Further, as shown in FIG. 3 and FIG. 4, a locking means 200 is provided in the cover 110 and the base 120 to prevent opening of the cosmetic case 100 when not in use. The locking means 200 may comprise of a fastener piece 200a on the cover 110 that matches a fastener unit 200b on the base 120 for securing purpose. Further, in alternate embodiments, a snap, a magnet or a button or any suitable locking means could be provided to secure the cosmetic case 100. The cover 110 and the base 120 may be formed of a suitable polymeric material such as acrylonitrile butadiene styrene or styrene acrylonitrile. In an alternative embodiment of the disclosure, the inside surface of the cover 110 may be provided with a mirror.

Further as shown in FIG. 6, the base 120 comprises a lower base 122 and an upper base 124 which together define an internal storage space 126 for housing the sanitizer 140, the wave deflector 150, at least one indicator 170a, 170b to indicate operation conditions of the sanitizer 140.

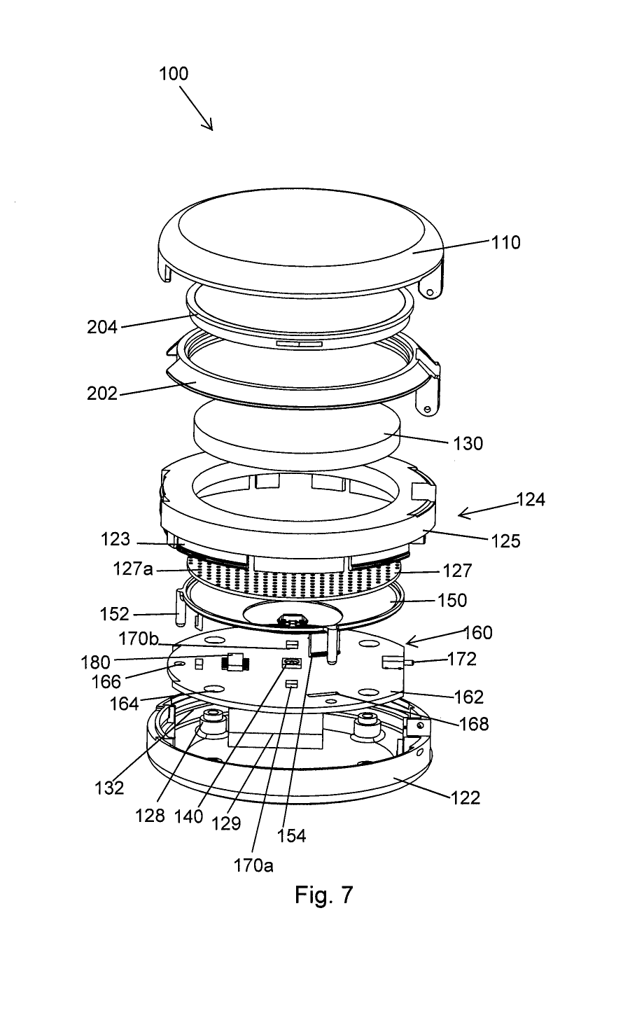

Further as shown in FIG. 6 and FIG. 7, the applicator 130 is stored in a cavity formed in the upper base 124. The upper base 124 comprises a side wall 125 and a bottom wall 127. As shown in FIG. 7, the bottom wall 127 is formed separately from the side wall 125 and then attached to a bottom end of the side wall 125. In alternate embodiments, the side wall 125 and the bottom wall 127 are made integrally as one unit. Further, at least a portion of the upper base 124 allows the ultra-violet rays emitted by the sanitizer 140 to pass through the internal storage space 126 to the applicator 130. As shown in FIG. 6 and FIG. 7, the bottom wall 127 has at least one aperture 127a to allow the ultra-violet rays emitted by the sanitizer 140 to pass through the internal storage space 126 to the applicator 130. According to an alternate embodiment, at least a portion of the upper base 124 is made of a material which allows ultra-violet rays emitted by the sanitizer 140 to pass through the internal storage space 126 to the applicator 130. According to an alternate embodiment, at least a portion of the upper base 124 is transparent or translucent to allow the ultra-violet rays emitted by the sanitizer 140 to pass through the internal storage space 126 to the applicator 130. According to another alternate embodiment, the bottom wall 127 is transparent or translucent to allow the ultra-violet rays emitted by the sanitizer 140 to pass through the internal storage space 126 to the applicator 130. According to an alternate embodiment, at least a portion of the upper base 124 is opaque with at least one aperture or micro perforation to allow the ultra-violet rays emitted by the sanitizer 140 to pass through the internal storage space 126 to the applicator 130. Further, according to the present embodiment and as shown in FIG. 6 and FIG. 7, the side wall 125 is made of transparent or translucent material or at least a portion of the side wall 125 is made of transparent or translucent material.

According to an embodiment, the applicator 130 may include an applying portion and a non-applying portion. Preferably, the applying portion faces towards the internal storage space 126 when the applicator 130 is stored in the cavity of the upper base 120. According to yet another embodiment of the present disclosure, the applicator 130 can be stored in the cavity of the upper base 120 only when its applying portion faces towards the internal storage space 126.

The upper base 124 is connected to the lower base 122 by suitable attachment means. As shown in FIG. 6 and FIG. 7, the upper base has at least one fitment projection 123 extending from the side wall 125 towards the lower base 122. The at least one fitment projection 123 fits into a corresponding fitment groove 132 in the lower base 122. According to alternate embodiment, the upper base 124 is connected to the lower base 122 by other attachment means including a threaded attachment, snap fit and the like.

The internal storage space 126 further comprises a source of power supply 160. The source of power supply 160 includes: a printed circuit board 162 which is stored in the internal storage space 126 and the sanitizer 140 is mounted on the printed circuit board 162. The printed circuit board 162 is assembled in the internal storage space 126 and on the lower base 122. As shown in FIG. 7, the printed circuit board 162 has at least one cut out 164 which fits into a corresponding at least one projection 128 present in the lower base 122. According to an alternate embodiment of the present disclosure, a printed circuit board cover may be combined to the internal storage space 126 to fix the printed circuit board 162 to the internal storage space 126. The printed circuit board 162 is further combined with the one or more indicators 170a, 170b which are provided to indicate operation conditions of the sanitizer 140. The indicators 170a, 170b are formed with a light emitting diode which are turned on during sanitization of the applicator 130 by the sanitizer 140 and are turned off after completion of the sanitization of the applicator 130. According to an embodiment, the light emitting diode includes one or more RGB LED which may be a pulsating or a flashing LED.

The internal storage space 126 further includes at least one battery 129 to supply power to the sanitizer 140. The at least one battery 129 is mounted under the printed circuit board 162 and may be one time use or rechargeable, replaceable or one time use. According to the present embodiment and as shown in FIG. 6, the base 122 further includes a USB port 134 for charging the at least one battery 129.

The wave deflector 150 is assembled on the printed circuit board 162 in the internal storage space 126. The wave deflector 150 has at least one projection 152 which is inserted into a corresponding at least one guide groove 166 on the printed circuit board 162. Further, the wave deflector 150 has at least one fitment projection 154 which fits into a corresponding at least one fitment groove 168 on the printed circuit board 162.

According to yet another embodiment of the present disclosure, the one or more indicators 170a, 170b on the printed circuit board 162 also indicate charging of the at least one battery 129. The one or more indicators 170a, 170b emit different colored lights during sanitization of the applicator 130, during charging of the battery 129 and when the charging of the battery 129 is complete. Alternatively, the internal storage space 126 may comprise a separate charging indicator which indicates charging of the battery 129. The charging indicator is also formed with a light emitting diode which is turned on to emit a colored light during charging of the battery and emit a different colored light when the charging of the battery is complete. The light emitting diode includes one or more RGB LED which may be a pulsating or a flashing LED.

The printed circuit board 162 is further provided with a power button 172 which is combined to the hinge 190 and pressed during opening and closing of the cover 110. The power button 172 is provided to actuate the sanitizer 140 emitting the ultraviolet rays. The sanitizer 140 is actuated for a predetermined amount of time controlled by the micro-controller 180 when the power button 172 is pressed during closing of the cover 110. The sanitizer 140 is off during opening of the cover 110. According to an alternate embodiment, the power button 172 may be replaced by a sensor which functions during opening and closing of the cover 110. According to an embodiment, the button/sensor is a concealed button/sensor so that it operates only on closing and opening of the cosmetic case 100 and thus the button/sensor cannot be fiddled by the user. The power button 172 can be a magnetic or a mechanical button.

After using the applicator 130, as the cosmetic case 100 is closed, the power button 172 is pressed by closing of the cover 110 which turns on the sanitizer 140 for a predetermined amount of time controlled by the micro-controller 180 and sanitization process is indicated by one or more indicators 170a, 170b which is further controlled by the micro-controller 180. The light emitted by one or more indicators 170a, 170b during sanitization process is diffused towards the upper base 124. Accordingly, the user can confirm operation conditions of the sanitization as light emitted during sanitization is visible to user's naked eyes through the transparent or translucent side wall 125 of the upper base 124. Thus, a sanitized applicator 130 will be available to the user during next use of the cosmetic case 100.

Although the foregoing is directed to embodiments of the present disclosure, other and further embodiments of the disclosure may be devised without departing from the basic scope thereof, and the scope thereof is determined by the claims that follow. Accordingly, the appended claims should be construed to encompass not only those forms and embodiments of the invention specifically described above, but to such other forms and embodiments as may be devised by those skilled in the art without departing from its true spirit and scope.

What is claimed is:

1. A cosmetic case comprising:
a cover;
a base, the base includes a lower base and an upper base;
an applicator stored in a cavity formed in the upper base;
wherein an internal storage space is defined between the lower base and the upper base;
wherein a sanitizer and a wave deflector are housed in the internal storage space in the lower base;
wherein the sanitizer emits ultraviolet rays for sanitizing the applicator; and
wherein at least a portion of the upper base allows the ultra-violet rays emitted by the sanitizer to pass through the internal storage space to the applicator.

2. The cosmetic case according to claim 1, wherein, the sanitizer emits UVC rays with wavelengths between 200-280 nanometers.

3. The cosmetic case according to claim 2, wherein, the sanitizer is surrounded by the wave deflector.

4. The cosmetic case according to claim 1, wherein the wave deflector is of a frustoconical shape comprising a reflective internal surface.

5. The cosmetic case according to claim 1, wherein at least a portion of the upper base is transparent or translucent to allow the ultra-violet rays emitted by the sanitizer to pass through the internal storage space to the applicator.

6. The cosmetic case according to claim 1, wherein the upper base comprises a side wall and a bottom wall and wherein the bottom wall has at least one aperture to allow the ultra-violet rays emitted by the sanitizer to pass through the internal storage space to the applicator.

7. The cosmetic case according to claim 1, wherein a printed circuit board is housed in the internal storage space and wherein the printed circuit board is attached to the lower base.

8. The cosmetic case according to claim 7, wherein the printed circuit board includes connected thereto a microcontroller, a battery, the wave deflector, and the sanitizer.

9. The cosmetic case according to claim 8, wherein the printed circuit board is combined with one or more indicators to indicate operation conditions of the sanitizer and wherein the one or more indicators are light emitting diodes which are turned on during sanitization of the applicator and are turned off after completion of the sanitization of the applicator.

10. The cosmetic case according to claim 9, wherein the light emitting diodes includes RGB LED and wherein RGB LED is selected from a group of LED consisting of non-pulsating LED, a pulsating LED and a flashing LED.

11. The cosmetic case according to claim 8, wherein the battery is mounted under the printed circuit board for supplying power to the sanitizer and wherein the battery is selected from one of a non-rechargeable battery and a rechargeable battery.

12. The cosmetic case according to claim 11, wherein a USB port for charging the rechargeable battery is included in the lower base.

13. The cosmetic case according to claim 8, wherein the printed circuit board includes at least one charging indicator to indicate charging of a battery.

14. The cosmetic case according to claim 8, wherein the printed circuit board is provided with a power button to actuate the sanitizer emitting the ultraviolet rays, and wherein the power button is combined to an attachment of the cover to the base so that the power button is pressed during closing of the cover.

15. The cosmetic case according to claim 8, wherein the wave deflector has at least one projection which is inserted into a corresponding at least one guide groove on the printed circuit board.

16. The cosmetic case according to claim 1, wherein the cosmetic case further comprises a lift tray for storing a cosmetic.

17. A cosmetic case comprising:
a cover;
a base, the base includes a lower base and an upper base,
an internal storage space defined between the lower base and the upper base;
a printed circuit board housed in the internal storage space;
a wave deflector, a sanitizer, a microcontroller, and a battery attached to the printed circuit board in the internal storage space;
wherein the sanitizer emits ultraviolet rays; and
wherein at least a portion of the upper base allows the ultra-violet rays emitted by the sanitizer to pass through.

18. The cosmetic case according to claim 17, wherein the sanitizer is actuated for a predetermined amount of time controlled by the micro-controller.

19. The cosmetic case according to claim 17, wherein the sanitizer is switched on when a power button of the cosmetic case is pressed by closing of the cover and wherein the sanitizer is switched off by opening of the cover.

20. A cosmetic case comprising:
a cover;
a base, the base includes a lower base and an upper base,
an internal storage space defined between the lower base and the upper base;
a printed circuit board housed in the internal storage space;
a sanitizer, a microcontroller, an indicator and a battery attached to the printed circuit board in the internal storage space;
wherein the sanitizer emits ultraviolet rays;
wherein at least a portion of the upper base allows the ultra-violet rays emitted by the sanitizer to pass through;
wherein at least a portion of the upper base is transparent or translucent; and
wherein an indicator light emitted during sanitization is visible to user's naked eyes through the transparent or translucent portion of the upper base.
